(19) United States
(12) Patent Application Publication
(10) Pub. No.: US 2007/0172833 A1
(43) Pub. Date: Jul. 26, 2007

(54) Gene expression profile retrieving apparatus, gene expression profile retrieving method, and program

(76) Inventors: Wataru Fujibuchi, Tokyo (JP); Paul Horton, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/235,150

(22) Filed: Sep. 27, 2005

(30) Foreign Application Priority Data
Sep. 27, 2004 (JP) 2004-280257

(51) Int. Cl. C12Q 1/68; G06F 19/00

(52) U.S. Cl. 435/6; 702/20

(57) ABSTRACT

In a gene expression profile retrieving apparatus, while gene expression profile data acquired with different platforms can be used, a cell can be retrieved based upon a gene expression profile. The gene expression profile retrieving apparatus is provided with: a gene expression profile DB stores gene expression profiles of known cells in a plurality of different platforms; a reference gene selecting unit operable to select a plurality of reference genes from genes which are commonly contained in both an inquiry profile and a platform of a gene expression profile stored in the gene expression profile DB; an order applying unit operable to apply orders to the inquiry profile and the reference gene of the known cell stored in the gene expression profile DB according to the expression level; and an analogous cell determining unit operable to acquire from the gene expression profile DB, such a cell which is analogous to the gene expression profile of the inquiry profile in the highest degree based upon the applied order.

### BACKGROUND OF THE INVENTION

1. Field of the Invention

The present invention relates to a gene expression profile retrieving apparatus capable of retrieving a cell from a database which stores gene expression profiles of known cells by using a gene expression profile as a key.

2. Background

Presently, gene expression monitoring researches with employment of DNA microarrays have been actively carried out. For instance, in “Tumorclassification and marker gene prediction by feature selection and fuzzy-means clustering using microarray data.” Wang J. Bo T H, Jonassen l, Myklebost O, Hovig B, BMC Bioinformatics. 2003 Dec. 2; 4(1):60, the research of seeking the genes which are different by using the gene expression profile data has been announced. Also, in “Multiclasscancer diagnosis using tumor gene expression signatures.” , Ramaswamy S, TamayoP, Rifkin R, Mukherjee S, Yeang CH, Angelo M, Ladd C, Reich M, Latulippe E, MesirovJP, Poggio T, Gerald W, Loda M, Lander E S, Golub T R, Proc Natl Acad Sci USA. 2001 Dec. 18; 98(26):15149-54, the research of seeking the cancers by using the gene expression profiles has been announced.

As previously described, as a result of actively progressing the researches of the gene expression monitoring with employment of the DNA microarrays, while huge amounts of data about gene expression profiles have been stored in universities, research institutions, and the like in the world. It is predictable that these gene expression profile data amounts may be increased in the future.

### SUMMARY OF THE INVENTION

If a cell may be specified by analyzing the gene expression profiles of the cell by DNA microarrays, it may become very useful in various fields such as pathological analyses, criminal investigations, and so on. In order to specify cells based upon gene expression profiles, a database of gene expression profiles covering all of patterns must be constructed. In other words, either a cell whose gene expression profile is identical or highly analogous to the gene expression profile of an unknown cell is retrieved from the gene expression profile database covering all type of cells, so that the unknown cell can be specified.

However, it is practically difficult that such a database is constituted by one research institution. As previously explained, since huge amounts of gene expression profiles have been stored in these research institutions in the world now, if these gene expression profile data are utilized, cells could be retrieved based upon the gene expression profiles. However, it should be understood that the below-mentioned difficulties may occur in order to realize such cell retrieving operations.

That is to say, a DNA microarray technique is an analyzing process that a marked nucleic acid (target) is hybridized to probe DNAs which have been aligned on abase board in high density, and then acquired images are captured by an automatic detector so as to be analyzed. A base board where probe DNAs have been aligned will be referred to as a platform. While platforms are different from each other due to differences in providers of DNA microarrays, since genes which will be hybridized are different from each other, gene expression profiles with different platforms of cells cannot be simply compared with each other. As described in the above-described publications, the present DNA microarray researches stop at such an experimental stage that, gene expression profiles acquired with the same platform are compared, but have not yet compared gene expression profiles with different platforms.

As previously explained, since there are differences in the platforms of the DNA microarrays, it is not so easy to mutually utilize gene expression profile data acquired with the different platforms. Under such a circumstance, it is practically difficult to constitute a consolidated database covering all type of cells, and also, cells have not been retrieved based upon gene expression profiles.

In consideration of above-described back ground, the present invention will provide a gene expression profile retrieving apparatus capable of using gene expression profile data with different platforms so as to retrieve cells based upon gene expression profiles.

A gene expression profile retrieving apparatus according to the present invention comprising: a gene expression profile database which stores the gene expression profile of known cells, wherein the profile data have been acquired with a plurality of different platforms; input section operable to accept an input of an inquiry profile indicative of a gene expression profile of a cell to be retrieved; reference gene selecting section operable to select a plurality of reference genes from plural genes which are commonly contained in both the platform of the inquiry profile and a platform of the gene expression profile stored in the gene expression profile database; order applying section operable to apply orders to the reference genes of the inquiry profile according to the expression level of each gene, and to apply orders to the reference genes of each cells stored in the gene expression profile database according to the expression level of each gene; analogous cell determining section operable to determine a cell from the plural cells stored in the gene expression profile database, wherein a combination of the orders applied to the respective reference genes of the cell to be determined is analogous to a combination of the orders applied to the reference genes of the inquiry profile in the highest degree; and output section operable to output the cell determined by the analogous cell determining section as a retrieved result.

A gene expression profile retrieving apparatus according to the present invention wherein the reference gene selecting section may subdivide the genes which constitute the inquiry profile into a plurality of groups according to the expression level of each gene, and selects at least one gene from each groups as the reference gene.

A gene expression profile retrieving apparatus according to the present invention wherein the reference gene selecting section may select a predetermined number of genes as the reference genes so that cells can be distinguished from each other based upon an analogous degree of the combinations of the orders.

A gene expression profile retrieving apparatus according to the present invention wherein: the reference gene selecting section may select 50 or more pieces of genes as the reference genes.

A gene expression profile retrieving apparatus according to the present invention wherein the analogous cell determining section may determine a plurality of cells in the order of higher analogous degrees between the combination of the orders applied to the respective reference genes of the cells which have been stored in the gene expression profile database, and the orders applied to the respective reference genes of the inquiry profile.

A gene expression profile retrieving method for retrieving a cell according to the invention, while a gene expression profile is employed as a key, from a gene expression profile database which stores the gene expression profile of known cells, wherein the profile data have been acquired with a plurality of different platforms, comprising: an input step for accepting an input of an inquiry profile indicative of a gene expression profile of a cell to be retrieved; a reference gene selecting step for selecting a plurality of reference genes from plural genes which are commonly contained in both a platform of the inquiry profile and a platform of a gene expression profile stored in the gene expression profile database; an order applying step for applying an orders to the reference genes of the inquiry profile according to the expression level of each gene, and for applying orders to the reference genes of each cells stored in the gene expression profile database according to the expression level of each gene; an analogous cell determining step for determining a cell from the plural cells stored in the gene expression profile database, wherein the orders applied to the respective reference genes of the cell to be determined is analogous to a combination of the orders applied to the reference genes of the inquiry profile in the highest degree; and an output step for outputting the cell acquired by the analogous cell determining step as a retrieved result.

A program for retrieving a cell according to the present invention, while a gene expression profile is employed as a key, from a gene expression profile database which stores the gene expression profile of known cells, wherein the profile data have been acquired with a plurality of different platforms, the program causes a computer to execute: an input step for accepting an input of an inquiry profile indicative of the gene expression profile of a cell to be retrieved; a reference gene selecting step for selecting a plurality of reference genes from plural genes which are commonly contained in both a platform of the inquiry profile and a platform of the gene expression profile stored in the gene expression profile database; an order applying step for applying an order to the reference gene of the inquiry profile according to the expression level of each gene, and for applying an order to the reference gene of each of the cells stored in the gene expression profile database according to the expression level of the gene; an analogous cell determining step for determining a cell from the plural cells stored in the gene expression profile database wherein combination of the orders applied to the respective reference genes of the cell to be determined is analogous to a combination of the orders applied to the reference genes of the inquiry profile in the highest degree; and an output step for outputting the cell determined by the analogous cell determining step as a retrieved result.

### BRIEF DESCRIPTION OF THE DRAWINGS

The accompanying drawings are incorporated in and constitute apart of this specification. The drawings exemplify certain aspects of the invention and, together with the description, serve to explain some principles of the invention.

FIG. 1 is a block diagram for schematically showing an arrangement of a gene expression profile retrieving apparatus according to an embodiment of the present invention;

FIG. 2A and FIG. 2B are diagrams for showing an example of data stored in a gene expression profile DB of the gene expression profile retrieving apparatus shown in FIG. 1;

FIG. 3 is a diagram for showing an example of actual data stored in the gene expression profile DB;

FIG. 4 is a diagram for showing an example of an inquiry profile;

FIG. 5 is a flow chart for showing operations of the gene expression profile retrieving apparatus of the present embodiment;

FIG. 6A and FIG. 6B are diagrams for showing orders which are applied by an order applying unit in the gene expression profile retrieving apparatus of the present embodiment;

FIG. 7 is a graphic diagram for graphically showing experimental results of retrieving operations executed by the gene expression profile retrieving apparatus of the embodiment;

FIG. 8 is a diagram for showing plotted the expression level with two different sorts of platforms;

FIG. 9 is a graphic diagram for graphically showing both a correlative coefficient and a rank correlative coefficient as to a total number of common genes and gene expression data between the different platforms;

FIG. 10 is a graphic diagram for graphically showing a result of an experiment capable of distinguishing a cancer cell from an ordinary cell by employing the rank correlative coefficient; and

FIG. 11 is a graphic diagram for graphically showing a result of an experiment for identifying a kidney cell from 16 sorts of cells.

### DETAILED DESCRIPTION

A gene expression profile retrieving apparatus according to the embodiment comprising: a gene expression profile database which stores the gene expression profile of known cells, wherein the profile data have been acquired with a plurality of different platforms; input section operable to accept an input of an inquiry profile indicative of a gene expression profile of a cell to be retrieved; reference gene selecting section operable to select a plurality of reference genes from plural genes which are commonly contained in both the platform of the inquiry profile and a platform of the gene expression profile stored in the gene expression profile database; order applying section operable to apply orders to the reference genes of the inquiry profile according to the expression level of each gene, and to apply orders to the reference genes of each cells stored in the gene expression profile database according to the expression level of each gene; analogous cell determining section operable to determine a cell from the plural cells stored in the gene expression profile database, wherein a combination of the orders applied to the respective reference genes of the cell to be determined is analogous to a combination of the orders applied to the reference genes of the inquiry profile in the highest degree; and output section operable to output the cell determined by the analogous cell determining section as a retrieved result.

The genes are selected which are commonly contained in the platform of the inquiry profile and the platform of the gene expression profile stored in the gene expression profile database, so that the gene expression profile retrieving apparatus can compare the gene expression profiles between the platforms where the probed genes are different from each other. Also, since the inquiry profile is compared with the gene expression profile of the gene expression profile database based upon the analogous degrees of the combinations of the orders as to the expression level of the reference genes, the gene expression profile retrieving apparatus can calculate the analogous degrees of the cells between such platforms in which the dynamic ranges of the expression level data, the resolution, and the S/N ratios are different. As a consequence, with employment of the arrangement, the cell which is analogous to the inquiry profile can be retrieved from the gene expression profile database which has stored the gene expression profiles of the cells with different platforms.

A gene expression profile retrieving apparatus according to the embodiment wherein the reference gene selecting section may subdivide the genes which constitute the inquiry profile into a plurality of groups according to the expression level of each gene, and selects at least one gene from each groups as the reference gene.

The reference genes are selected from the respective plural groups which have been subdivided according to the expression level of each gene, so that the gene expression profile retrieving apparatus can thoroughly select reference genes from the gene having the large order of the expression level up to the gene having the small order of the expression level. It should be noted that the group subdivision may be carried out according to magnitudes of the expression level of each gene, or may be performed according to orders of the expression level of each gene.

A gene expression profile retrieving apparatus according to the embodiment wherein the reference gene selecting section may select a predetermined number of genes as the reference genes so that cells can be distinguished from each other based upon an analogous degree of the combinations of the orders.

When a plurality of genes are selected as the reference genes, the total number of which covers the proper range where the cells can be identified. Therefore, the retrieving precision of the cell can be improved.

A gene expression profile retrieving apparatus according to the embodiment wherein: the reference gene selecting section may select 50 or more pieces of genes as the reference genes.

The inventors found out a knowledge that as to 50 or more pieces of genes, if combinations of orders according to the expression level are coincident with each other, a cell can be specified, and thus, could realize an apparatus, capable of performing a high precision retrieving operation by employing an arrangement that 50 pieces, or more pieces of reference genes are employed based upon this knowledge.

A gene expression profile retrieving apparatus according to the embodiment wherein the analogous cell determining section may determine a plurality of cells in the order of higher analogous degrees between the combination of the orders applied to the respective reference genes of the cells which have been stored in the gene expression profile database, and the orders applied to the respective reference genes of the inquiry profile.

The plurality of cells having the gene expression profiles highly analogous to the inquiry profile are retrieved, so that the most proper cell can be acquired from the outputted retrieved results.

A gene expression profile retrieving method for retrieving a cell according to the embodiment, while a gene expression profile is employed as a key, from a gene expression profile database which stores the gene expression profile of known cells, wherein the profile data have been acquired with a plurality of different platforms, comprising: an input step for accepting an input of an inquiry profile indicative of a gene expression profile of a cell to be retrieved; a reference gene selecting step for selecting a plurality of reference genes from plural genes which are commonly contained in both a platform of the inquiry profile and a platform of a gene expression profile stored in the gene expression profile database; an order applying step for applying an orders to the reference genes of the inquiry profile according to the expression level of each gene, and for applying orders to the reference genes of each cells stored in the gene expression profile database according to the expression level of each gene; an analogous cell determining step for determining a cell from the plural cells stored in the gene expression profile database, wherein the orders applied to the respective reference genes of the cell to be determined is analogous to a combination of the orders applied to the reference genes of the inquiry profile in the highest degree; and an output step for outputting the cell acquired by the analogous cell determining step as a retrieved result.

With employment of the above-explained profile retrieving method, similar to the gene expression profile retrieving apparatus of the embodiment, the cell which is analogous to the inquiry profile can be retrieved from the gene expression profile database which has stored the gene expression profiles of the cells with the plural different platforms. Also, the various sorts of arrangements of the gene expression profile retrieving apparatus according to the embodiment may be applied to the gene expression profile retrieving method according to the embodiment.

A program for retrieving a cell according to the embodiment, while a gene expression profile is employed as a key, from a gene expression profile database which stores the gene expression profile of known cells, wherein the profile data have been acquired with a plurality of different platforms, the program causes a computer to execute: an input step for accepting an input of an inquiry profile indicative of the gene expression profile of a cell to be retrieved; a reference gene selecting step for selecting a plurality of reference genes from plural genes which are commonly contained in both a platform of the inquiry profile and a platform of the gene expression profile stored in the gene expression profile database; an order applying step for applying an order to the reference gene of the inquiry profile according to the expression level of each gene, and for applying an order to the reference gene of each of the cells stored in the gene expression profile database according to the expression level of the gene; an analogous cell determining step for determining a cell from the plural cells stored in the gene expression profile database, wherein combination of the orders applied to the respective reference genes of the cell to be determined is analogous to a combination of the orders applied to the reference genes of the inquiry profile in the highest degree; and an output step for outputting the cell determined by the analogous cell determining step as a retrieved result.

With employment of the above-explained profile retrieving program, similar to the gene expression profile retrieving apparatus of the embodiment, the cell which is analogous to the inquiry profile can be retrieved from the gene expression profile database which has stored the gene expression profiles of the cells in the plural different platforms. Also, the various sorts of arrangements of the gene expression profile retrieving apparatus according to the embodiment may be applied to the gene expression profile retrieving program according to the embodiment.

Referring now to drawings, a gene expression profile retrieving apparatus according to an embodiment of the present invention will be described.

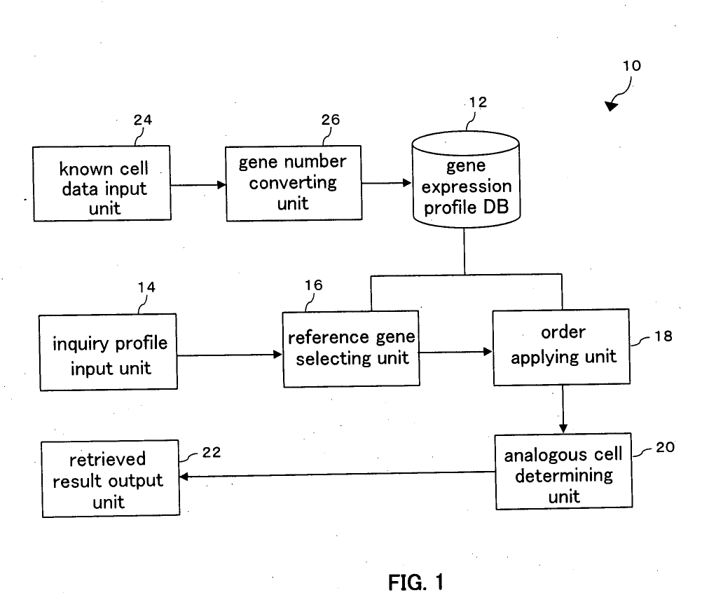

FIG. 1 is a schematic block diagram showing an arrangement of the gene expression profile retrieving apparatus 10 according to the present embodiment. The gene expression profile retrieving apparatus 10 is equipped with a gene expression profile database (will be referred to as “gene expression profile DB” hereinafter) 12, an inquiry profile input unit 14, and a retrieved result output unit 22 for outputting a retrieved result. The gene expression profile DB 12 has stored the gene expression profiles of known cells. The inquiry profile input unit 14 accepts an input of an inquiry profile which indicates a gene expression profile of a cell to be retrieved. The retrieved result output unit 12 outputs retrieved results.

Also, the gene expression profile retrieving apparatus 10 is equipped with a reference gene selecting unit 16, an order applying unit 18, and an analogous cell determining unit 20. These units 16,18,20 are used in order to retrieve a cell which has a gene expression profile highly analogous to an inquiring profile input by the inquiry profile input unit 14 from the gene expression profile DB 12.

The gene expression profile retrieving apparatus 10 is arranged by a normal computer equipped with a CPU (central processing unit), RAM (random access memory), a ROM (read-only memory), a display, a keyboard, and the like. The gene expression profile retrieving apparatus 10 executes a process operation in accordance with a program stored in the ROM so as to retrieve a cell from the gene expression profile DB 12 by using a gene expression profile as a key.

Now, the respective structural units employed in the gene expression profile retrieving apparatus 10 according to the present embodiment will be explained. The gene expression profile DB 12 has stored gene expression profiles of known cells. The gene expression profile DB 12 stores gene expression profile data acquired with a plurality of different platforms.

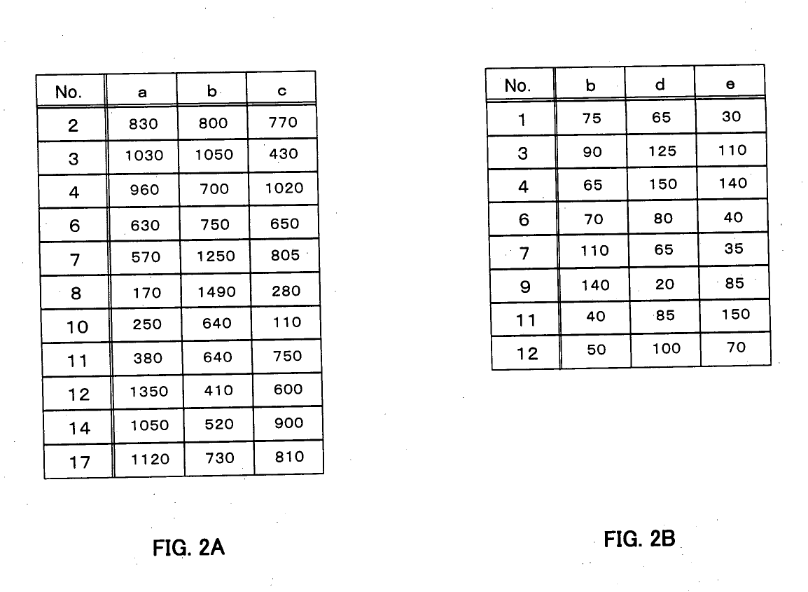

FIG. 2A and FIG. 2B are diagrams showing an example of the gene expression profile data with the different platforms stored in the gene expression profile DB 12. In the below-mentioned explanations, the platform of the gene expression profile shown in FIG. 2A will be referred to as a “platform A”, the platform of the gene expression profile indicated in FIG. 2B will be referred to as a “platform B.” The gene expression profile data of the platform A contains data as to the expression level of each gene, the gene numbers of which are 2, 3, 4, 6, 7, 8, 10, 11, and 12. In the platform A, the data as to the expression level of the genes have been acquired in the resolution of 0 to 1,500. The gene expression profile data of the platform B contains data as to the expression level of each gene, the gene numbers of which are 1, 3, 4, 6, 7, 9, 11, and 12. In the platform B, the data as to the expression level of the genes have been acquired in the resolution of 0 to 150. As can been seen from FIG. 2A and FIG. 2B, if a platform is different from another platform, sorts of genes which are hybridized, resolution and the like are different. It should be understood that three or more sorts of platforms may be employed, although this example shows that two sorts of the platforms A and B.

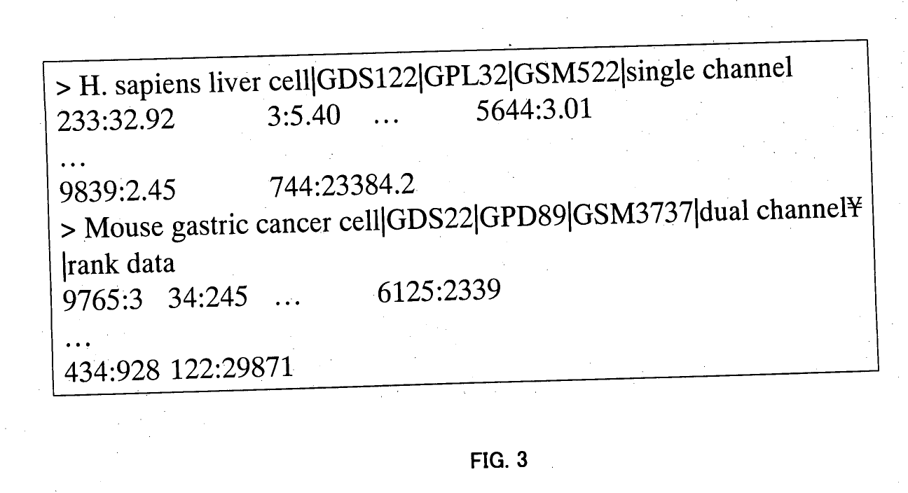

FIG. 3 is a diagram showing an example of actual data stored in the gene expression profile DB 12. In this example, an entry of gene expression profile data of each cells starts with such a symbol “>”, and a sort of the cell, a tissue name thereof, and also, a comment on the cell are described. Then, subsequent to a line feed, a gene number of a hybridized gene and the expression level are described. An actual gene number employed in the gene expression profile DB 12 is the gene number of UniGene. When specific gene numbers are employed in respective plural different platforms, after these specific gene numbers are converted into gene numbers of UniGene, the UniGene-converted gene numbers a restored in the gene expression profile DB 12. In order to perform such a gene number conversion, as shown in FIG. 1, the gene expression profile retrieving apparatus 10 is equipped with a gene number converting unit 26. This gene number converting unit 26 converts a gene number inputted from the known cell data input unit 24 into a gene number of UniGene.

The inquiry profile input unit 14 functions to accept an input of an inquiry profile indicative of a gene profile of a cell to be retrieved. The hardware of the inquiry profile input unit 14 is arranged by, for example, a data reading apparatus which reads out an inquiry profile from a recording medium on which an inquiry profile is recorded.

The reference gene selecting unit 16 functions to select a plurality of reference genes from genes which are commonly contained in both a platform of the inquiry profile input from the inquiry profile input unit 14, and the platform of the gene expression profile stored in the gene expression profile DB 12. An example for selecting reference genes from the commonly contained genes between an inquiry profile and the gene expression profile (refer to FIG. 2A) of the platform A will now be explained.

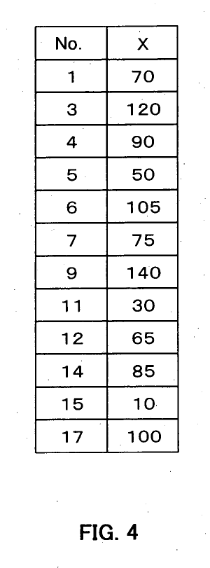

FIG. 4 is a diagram showing an example of an inquiry profile X. The inquiry profile X contains data of the expression level of genes, the gene numbers of which are 1, 3, 4, 5, 6, 7, 9, 11, 12, 14, 15, and 17. Firstly, the reference gene selecting unit 16 acquires genes which are commonly contained in both the platform of the inquiry profile X shown in FIG. 4 and the platform A stored in the gene expression profile DB 12, indicated in FIG. 2A. 8 pieces of genes whose gene numbers are 3, 4, 6, 7, 11, 12, 14, and 17 are commonly contained in both the inquiry platform X and the platform A. Next, the reference gene selecting unit 16 selects a plurality of genes from the genes which are commonly contained as the reference genes. At this time, in order to thoroughly select genes from a gene having a large order of the expression level up to a gene having a small order of the expression level, the reference gene selecting unit 16 subdivides the genes which are commonly contained in the inquiry platform X and the platform A into three groups according to orders of the expression level of genes in the inquiry profile, and then, selects at least one data from each groups. Concretely speaking, assuming that the commonly contained genes are subdivided into three groups: a first group includes genes that orders of the expression level are a first order to a fourth order; a second group includes genes that orders of the expression level are a fifth order to an eighth order; and a third group includes genes that orders of the expression level are a ninth order to a 12th order, the gene numbers of 3, 6, 17 among the above-explained commonly contained 8 genes are contained in the first group; the gene numbers of 4, 7, 14 are contained in the second group; and the gene numbers of 11, 12 are contained in the third group. Then, the reference gene selecting unit 16 selects at least one data from each group. Preferably, the reference gene selecting unit 16 may select equal quantities of genes from each group. For instance, the reference gene selecting unit 16 selects the genes of number 3, 17 from the first group; the genes of number 4, 7 from the second group; and the genes of number 11, 12 from the third group. For the sake of an easy explanation, this embodiment has been explained by using the data, the total gene number is small. However, in an actual case, the reference gene selecting unit 16 selects 50 or more pieces of genes as the reference genes from gene expression profile constituted by several thousands of genes up to several ten thousands of genes.

Referring back to FIG. 1, the order applying unit 18 functions to apply orders to both the reference gene of the inquiry profile X and the reference gene of each of cells stored in the gene expression profile DB 12 according to the expression level. In this embodiment, an example that the order applying unit 18 applies an order according to the expression level of the inquiry profile X will be explained. For instance, it is assumed that the genes of number 3, 4, 7, 11, 12, 17 are selected as the reference genes from the genes commonly contained between the inquiry profile X (refer to FIG. 4) and the gene expression profile of the platform A (refer to FIG. 2A). In this case, by referring to the expression level of the inquiry profile X, the order applying unit 18 applies a first order to the gene of number 3 whose expression level is 120; applies a second order to the gene of number 17 whose expression level is 100; applies a third order to the gene of number 4 whose expression level is 90; applies a fourth order to the gene of number 7 whose expression level is 75; applies a fifth order to the gene of number 12 whose expression level is 65; and applies a sixth order to the gene having the gene number 11 whose expression level is 30. The order applying unit 18 similarly applies orders to the reference genes of cells stored in the gene expression profile DB 12.

The analogous cell determining unit 20 functions to acquire a cell having a gene expression profile which is analogous to an inquiry profile based upon an order applied to a reference gene. The analogous cell determining unit 20 firstly calculates a rank correlative coefficient indicative of analogous degrees between a combination of orders applied to the reference genes of the inquiry profile and a combination of orders applied to the reference genes of the respective cells stored in the gene expression profile DB 12. The rank correlative coefficient “r” may be calculated based upon the following formula (1), while rank differences of the reference genes 1 to n are assumed as “Di”:

$$[ Formula 1 ] \\ r = 1 - \frac{6 {\sum }_{i = 1}^{n} {D}_{i}^{2}}{n ( n + 1 ) ( n - 1 )} \qquad ( 1 )$$

Subsequently, in order to calculate a significance of the rank correlative coefficient “r”, the analogous cell determining unit 20 calculates a t-distribution representing a difference with respect to a null hypothesis in which the rank coefficient is equal to 0 based upon the following formula (2):

$$[ Formula 2 ] \\ t = \frac{r \sqrt{n - 2}}{\sqrt{1 - {r}^{2}}} \qquad ( 2 )$$

Since the significance of the rank correlative coefficient is calculated, even when total numbers of the reference genes are different between the platform A and the platform B, analogous degrees can be properly calculated. Then, the analogous cell determining unit 20 determines a cell having a high significance as an analogous cell based upon the t-distribution.

The retrieved result output unit 22 functions to output a cell determined by the analogous cell determining unit 20 as a retrieved result. The hardware of the retrieved result output unit 22 is constituted by, for example, a display, a printer, or the like.

Next, operations of the gene expression profile retrieving apparatus 10 according to the present embodiment will be explained. In the below-mentioned example, the gene expression profile retrieving apparatus 10 retrieves a cell having the inquiry profile X shown in FIG. 4 from the gene expression profile DB 12 by.

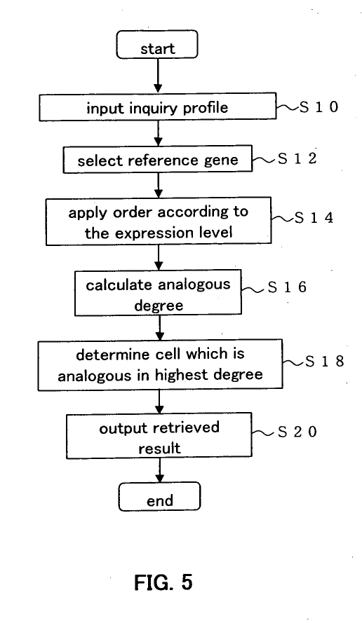

FIG. 5 is a flow chart showing process operations of the gene expression profile retrieving apparatus 10 according to the present embodiment. First, the inquiry profile input unit 14 accepts an input of the inquiry profile X shown in FIG. 4 (step S10). Concretely speaking, the inquiry profile X is input to the gene expression profile retrieving apparatus 10 by reading the recording medium, on which the inquiry profile X has been recorded, by the gene expression profile retrieving apparatus 10.

When the input of the inquiry profile X is accepted by the inquiry profile input unit 14, the reference gene selecting unit 16 selects a plurality of reference genes from the genes commonly contained in both the platform of the inquiry profile X and the platform of the gene expression profile stored in the gene expression profile DB 12 (step S12). For example, the reference gene selecting unit 16 selects the genes of number 3, 4, 7, 11, 12, 17 as the reference from the gene expression profile (refer to FIG. 2A) of the platform A, and also selects the genes of number 1, 4, 6, 9, 11, 12 as the reference genes from the gene expression profile (refer to FIG. 2B) of the platform B. It should also be understood that same reference genes may be selected with respect to the platform A and the platform B.

Next, the order applying unit 18 applies orders to both the reference genes of the inquiry profile X and the reference genes of the respective cells stored in the gene expression profile DB 12 according to the expression level (step S14).

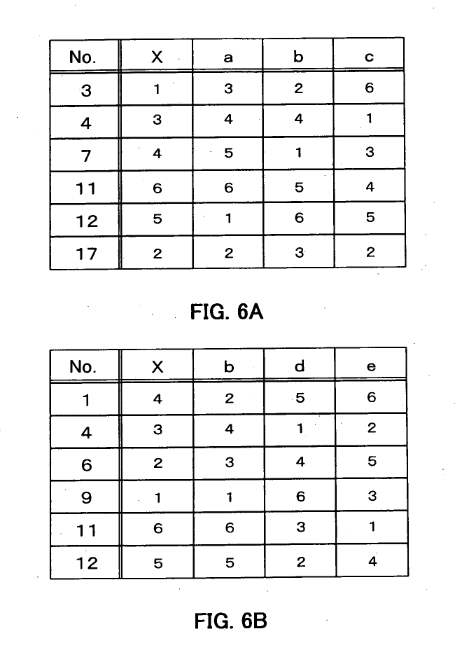

FIG. 6A is a diagram showing orders which have been applied to both the inquiry profile X and reference genes of the gene expression profile of the platform A, and FIG. 6B is a diagram showing orders applied to both the inquiry profile. X and reference genes of the gene expression profile of the platform B. For example, as indicated in FIG. 6A, the orders are applied to both the inquiry profile X and the reference genes of the respective cells “a”, “by”, “c” of the gene expression profile DB 12. Since the orders are applied according to the expression level, the cells acquired with the different platforms can be compared with each other.

Subsequently, the analogous cell determining unit 20 calculates an analogous degree between the inquiry profile X and a gene expression profile of a cell stored in the gene expression profile DB 12 based upon a combination of the orders applied by the order applying unit 18 (step S16). Concretely speaking, the analogous cell determining unit 26 calculates a rank correlative coefficient between an order applied to each of the reference genes of the inquiry profile X and each of the reference genes of the cells stored in the gene expression profile DB 12 based upon the above-described formula (1). A calculation example of a rank correlative coefficient between the inquiry profile X and the cell “a” of the platform A is explained. Since a rank difference of the gene number 3 is equal to 2, a rank difference of the gene numbers 4 and 7 is equal to 1, a rank difference of the gene number 12 is equal to 4, and also, a rank difference of the gene numbers 11 and 17 is equal to 0, a term of SD² becomes 2²+1²+1²+0²+4²+0²=22. Since the number “n” of the reference gene is equal to 6, the rank correlative coefficient “r” is as follows: r=1−6×SD²/(n(n+1) (n−1)=1−6×22/(6×7×5)=0.37. Subsequently, the analogous cell determining unit 20 calculates a t-distribution by substituting the calculated rank coefficient for the above-described formula (2). The significance indicated by this t-distribution expresses analogous degree of the cell. In this embodiment, since the t-distribution indicative of the significance is calculated by using the rank correlative coefficient “r” and the reference gene number “n” as parameters, even when the total numbers of reference genes are different from each other which are selected from a plurality of different platforms, analogous degrees may be properly compared with each other.

Next, the analogous cell determining unit 20 determines a cell which is analogous to the inquiry profile X in the highest degree from the cells stored in the gene expression profile DB 12 based upon the calculated analogous degrees (step S18). The analogous cell determining unit 20 determines a cell having the highest significance as the cell having the highest analogous degree. Alternatively, the analogous cell determining unit 20 may judge a cell having the highest significance is not analogous cell when the rank correlative coefficient of the cell does not exceed a predetermined threshold value. In this case, the analogous cell determining unit 20 judges that there is no cell corresponding to the inquiry profile X.

Next, the retrieved result output unit 22 outputs the cell determined by the analogous cell determining unit 20 as a retrieved result (step S20). Both the arrangement and the operations of the gene expression profile retrieving apparatus 10 according to this embodiment have been so far described.

Subsequently, experimental results obtained from the retrieving operations executed by the gene expression profile retrieving apparatus 10 according to this embodiment will be explained. An experimental condition of an experiment using the gene expression profile retrieving apparatus 10 is as follows: That is, firstly, gene expression profile data of 823 pieces of cells have been stored in the gene expression profile DB 12. These 823 cells contain 5 pieces of liver cells. While a liver cell is employed as a cell which should be retrieved, a gene expression profile of the liver cell is input as an inquiry profile. As a consequence, when any one of the 5 liver cells contained in the gene expression profile DB 12 is outputted as the retrieved result, this retrieved cell is a correct answer. In this experiment, while a total number of genes which are selected as reference genes is changed, a change in a correct answering rate caused by the change in the reference gene numbers was investigated. The retrieving operations were carried out 100 times while the gene to be selected as the reference gene is changed as to each of the gene numbers, a total number when the correct retrieved results could be obtained was divided by a total experiment execution time (100 times) so as to calculate a correct answering rate.

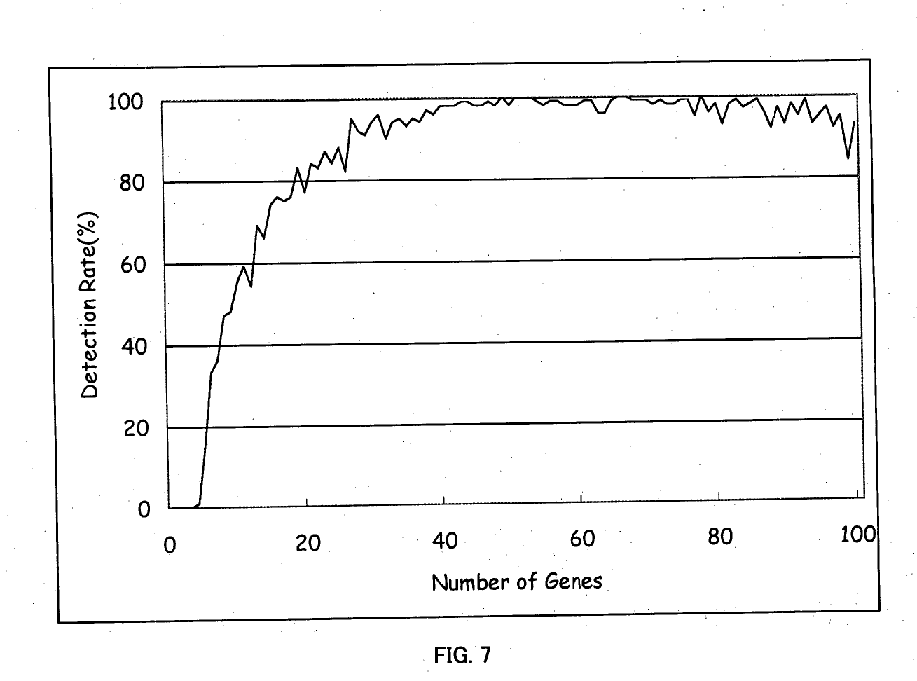

FIG. 7 is a diagram for representing retrieved results made by the gene expression profile retrieving apparatus 10 according to the present embodiment. In FIG. 7, the abscissa shows a total number of genes which are selected as reference genes, and the ordinate shows a rate at which correct answers are obtained by performing experiments 100 times. As can be seen from the experimental results indicated in FIG. 7 revealed that the correct answering rate may be almost equal to 100% when 50 or more pieces of genes are selected as the reference genes. As a result, in order to retrieve cells in high precision, it is preferable to select 50 or more pieces of reference genes. The experimental results obtained by the gene expression profile retrieving apparatus 10 according to the present embodiment have been so far explained.

The gene expression profile retrieving apparatus 10 according to the present embodiment selects as the reference genes, genes commonly contained in the platform of the inquiry profile and the platform of the gene expression profile stored in the gene expression profile DB 12, so that even when the platform of the inquiry profile is different from the platform of the inquiry expression profile of the gene expression profile DB 12, the gene expression profile retrieving apparatus 10 can compare the gene expression profiles with each other by employing the reference genes.

Also, the gene expression profile retrieving apparatus 10 according to the present embodiment retrieves a cell which is analogous to the inquiry profile from the gene expression profile DB 12 based upon the analogous degrees which is calculated by employing the orders applied to the respective reference genes according to the expression level. As a consequence, the gene expression profile retrieving apparatus 10 can calculate the analogous degrees between the cells with different platforms that the dynamic ranges of the expression level, the resolution, and the S/N ratios are different from each other.

Now, an effect which may be achieved by judging the analogous degrees in a manner that the orders are applied to the reference genes according to the expression level, and by employing the rank correlative coefficient of the applied orders will be explained by using concrete data.

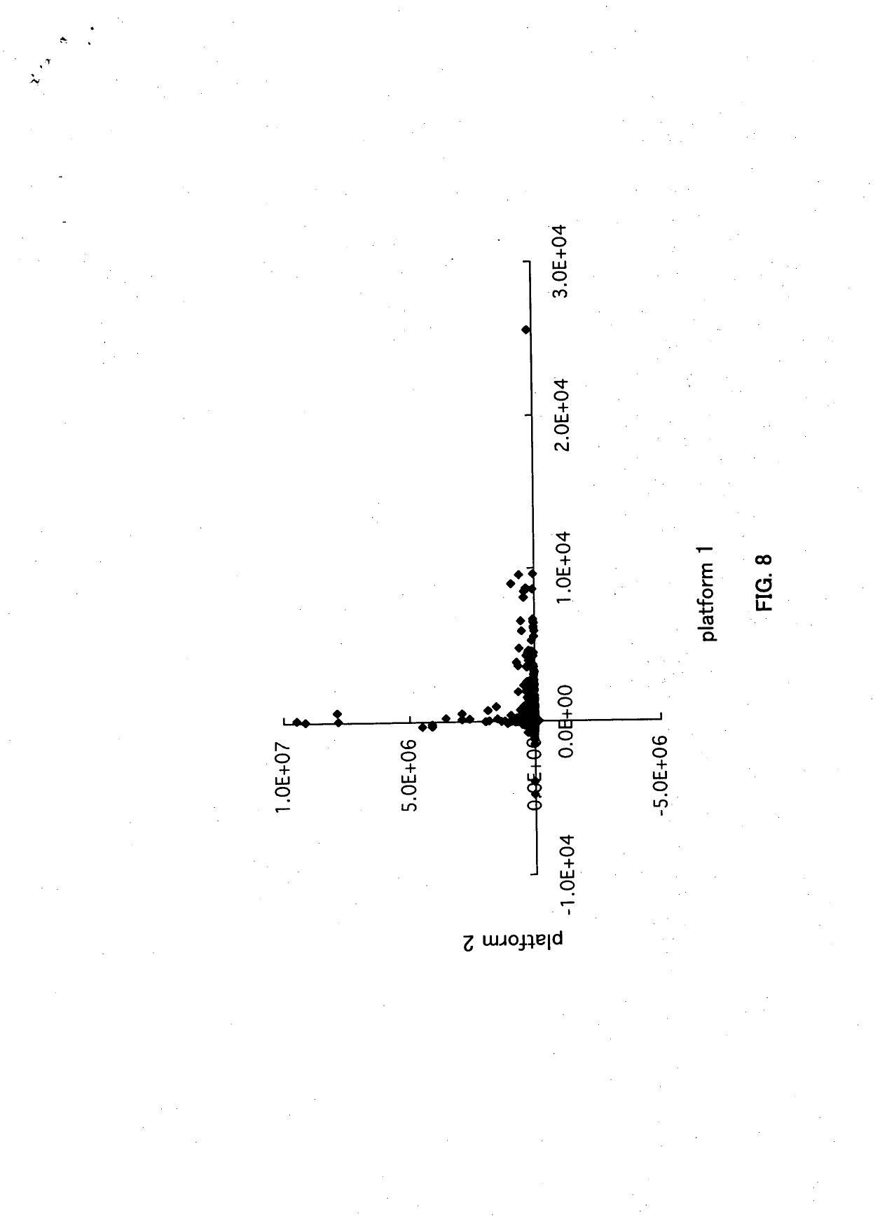

FIG. 8 is a graphic diagram showing that a liver cell of a human is hybridized to DNA microarrays of two different sorts of platforms, and then, the expression level in respective platforms of 3,050 pieces of genes which are commonly contained in the DNA microarrays are plotted. Referring to FIG. 8, it can be understood that the plotted data are widely distributed, measurement values of the expression level are distorted by each of the platforms. In other words, FIG. 8 teaches the gene expression profiles may not be properly compared with each other by using the normal correlative coefficients which uses measurement values themselves of the expression level are employed among the different platforms.

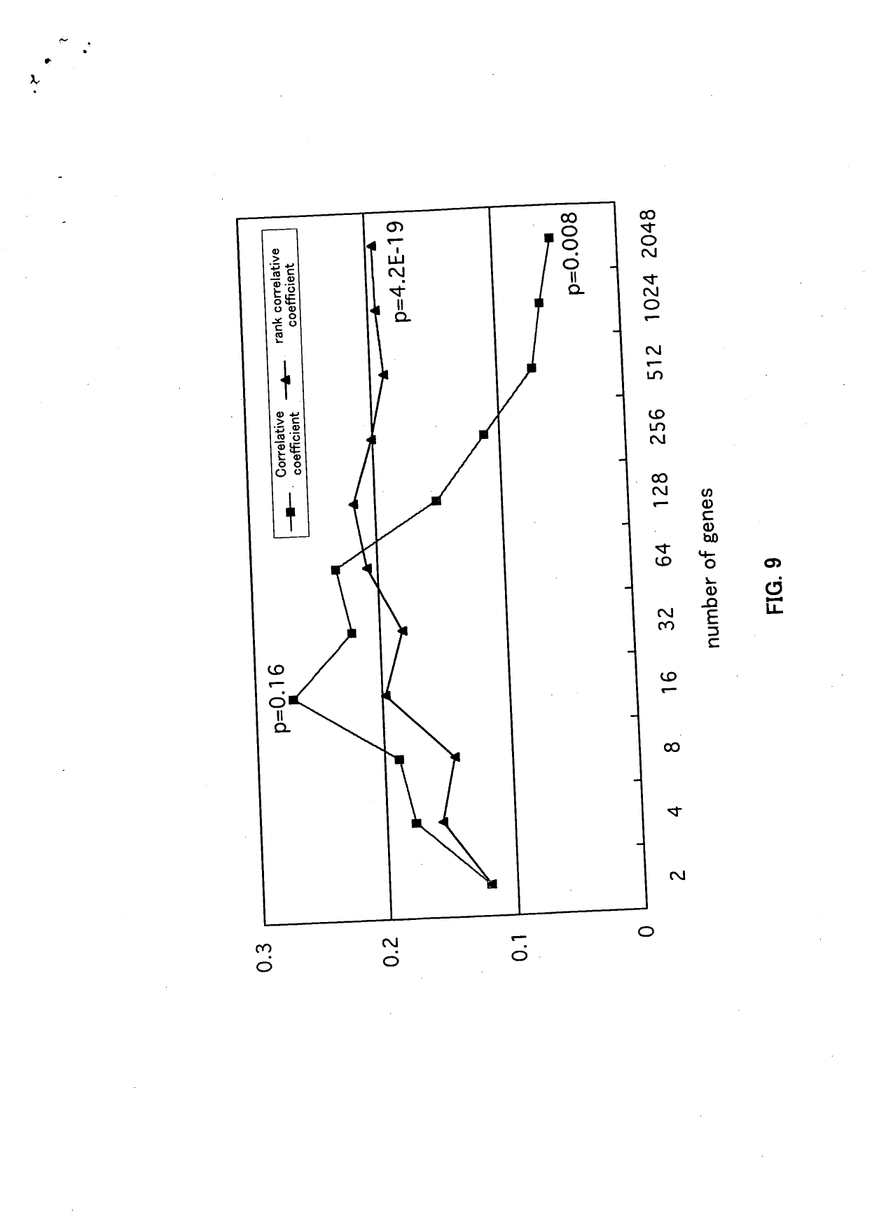

FIG. 9 is a graphic diagram for representing both a normal correlative coefficient and a rank correlative coefficient of gene expression data, and a common gene number between different platforms. A liver cell was hybridized to DNA microarrays of different platforms, and both correlative coefficients and rank correlative coefficients of gene expression profiles between the different platforms were calculated. Each point shown in FIG. 9 corresponds to an average value of experimental results performed 100 times is plotted. In FIG. 9, the abscissa indicates a total number of reference genes which are used in order to calculate a coefficient or a rank correlative coefficient, and the ordinate indicates either the correlative coefficient or the rank correlative coefficient. In this graphic diagram, the “correlative coefficient” corresponds to such a correlative coefficient which uses measurement values themselves of the expression level, and the “rank correlative coefficient” corresponds to such a rank correlative coefficient of orders which are applied to genes according to measurement values of the expression level. FIG. 9 shows the correlative coefficient calculated by using the measurement data of the expression level is changed, depending upon the reference gene number, whereas the rank correlative coefficient becomes stable irrespective of the reference gene number. When the number of genes which are used is increased, then the reliability in the “t”-investigation is increased. For example, when 2,004 pieces of genes were employed and the rank correlative coefficient were employed, p=4, and 2E-19. Thus, the significance could be clearly represented, as compared with 0.008 obtained by the normal correlative coefficient. As apparent from the above-described fact, when the gene expression profiles between the different platforms are compared, then it can be seen that the stronger correlation may appear in the rank correlative coefficient rather than the correlative coefficient.

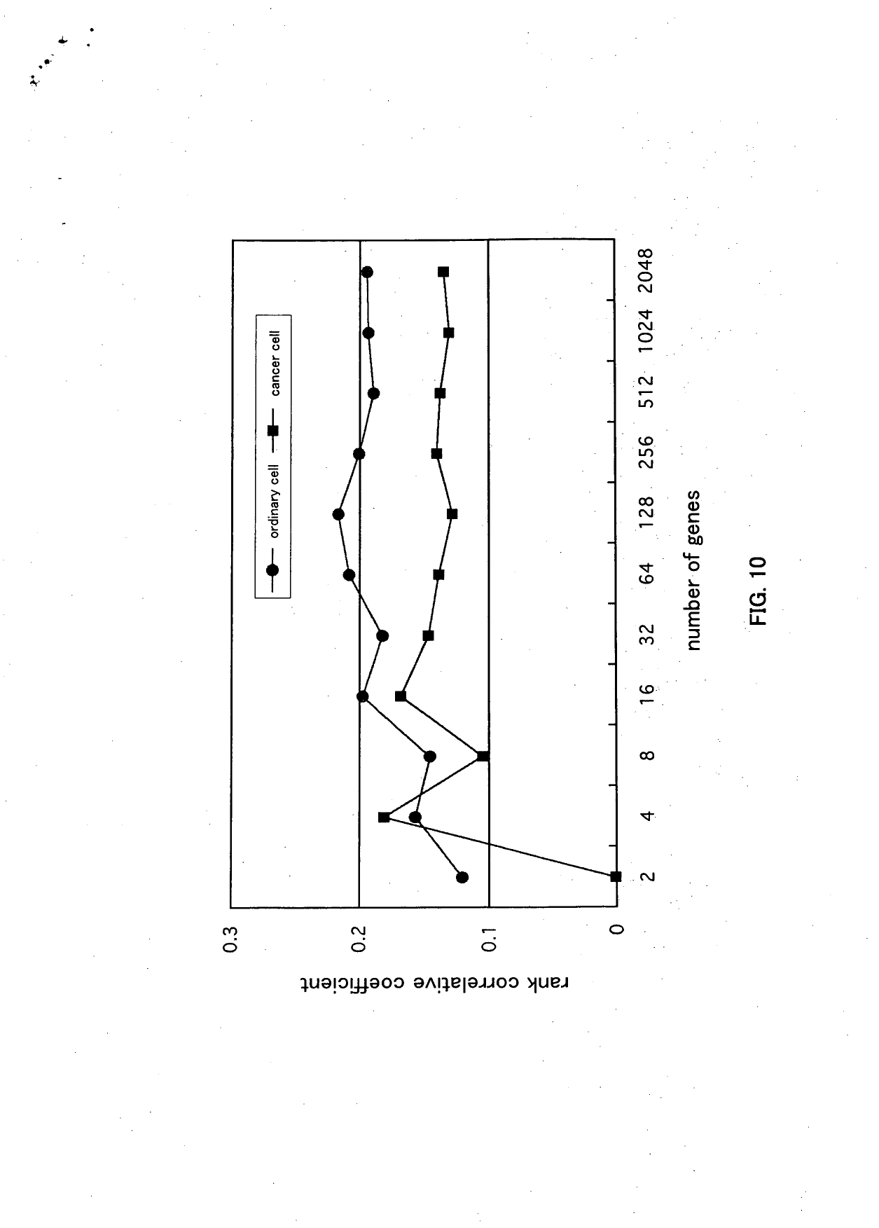

FIG. 10 is a graphic diagram for indicating a result of an experiment of distinguishing a cancer cell from an ordinary cell by applying a rank correlative coefficient to different platforms. Gene expression profiles of cancer cells and ordinary cells have been stored in a gene expression profile DB for known cells. Then, while an inquiry profile of an ordinary cell different from the platform of the gene expression profile DB is employed, rank correlative coefficients with respect to the respective cells of the gene expression profile DB are calculated. Each point shown in FIG. 10 corresponds to an average value as to experimental results performed 100 times is plotted.

As indicated in FIG. 10, in the case that the ordinary cells were compared with each other, the rank correlative coefficient was maintained at approximately 0.2 irrespective of the number of reference genes, whereas in the case that the ordinary cell was compared with the cancer cell, the rank correlative coefficient became on the order of 0.13, so that a significant difference could been seen between these rank correlative coefficients. Under such a circumstance, it can be understood that the cancer cell can be distinguished from the ordinary cell based upon the rank correlative coefficients.

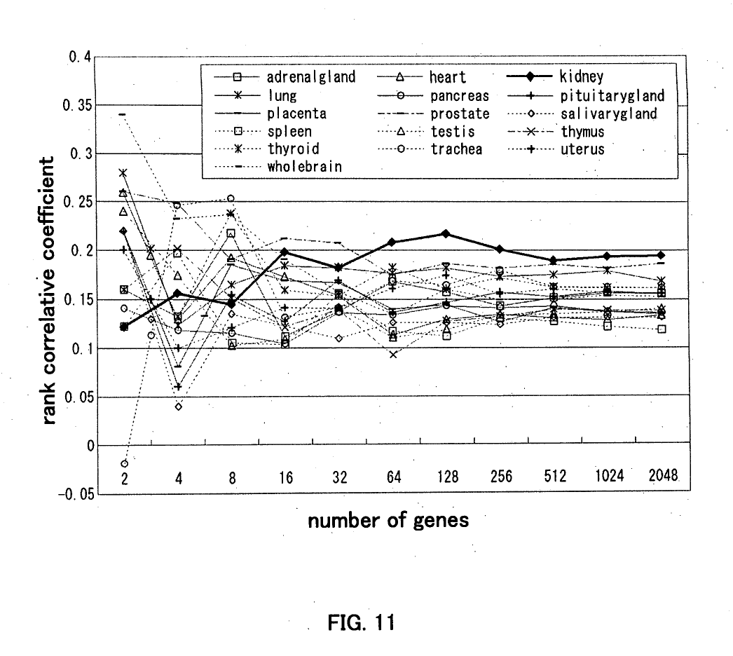

FIG. 11 is a graphic diagram for indicating results of experiments of identifying a kidney cell from 16 sorts of cells on different platforms. Gene expression profiles as to the 16 sorts of cells including the kidney cell have been stored in the gene expression profile DB for the known cells. Then, using an inquiry profile of a kidney cell on different platforms from the gene expression profile DB, rank correlative coefficients between the inquiry profile and the each cell of the gene expression profile DB are calculated. Each point shown in FIG. 11 corresponds to an average value as to experimental results performed 100 times is plotted.

As shown in FIG. 11, in case that a total number of the reference genes became larger than or equal to 64, then a rank correlative coefficient of the kidney cell which is the same as the inquiry profile among the 16 sorts of cells was stably increased, so that a stable difference between the rank correlative coefficient of the kidney cell and the rank correlative coefficients of other cells could be seen. This fact reveals that the kidney cell can be identified from the 16 sorts of cells based upon the rank correlative coefficients.

As previously explained, even when the gene expression profiles acquired with the different platforms are compared and this comparing operation can be hardly carried out by employing the measurement values themselves of the expression level data, the cells can be properly compared by using the rank correlative coefficients. In this embodiment, the orders are applied to the reference genes according to the expression level, and then, the analogous degrees are calculated based upon the rank correlative coefficients of the applied orders, so that the cells can be compared with each other between the different platforms.

Also, the gene expression profile retrieving apparatus 10 of the present embodiment subdivides the genes commonly contained in both the platform of the inquiry profile and the platform of the gene expression profile stored in the gene expression profile DB 12 into the plural gene groups according to the orders of the expression level in the inquiry profile, and then, selects as the reference gene at least one piece of gene from each gene group. As a result, the gene expression profile retrieving apparatus 10 can thoroughly select reference genes from the gene having the large order of the expression level up to the gene having the small order of the expression level, and thus, can retrieve the gene expression profile in the higher precision.

Although the gene expression profile retrieving apparatus 10 of the present invention has been described in detail by exemplifying the embodiment, the present invention is not limited only the above-described embodiment.

In the above-explained embodiment, the gene expression profile retrieving apparatus 10 is arranged by employing the single computer. However, the gene expression profile retrieving apparatus 10 need not be arranged by using such a single computer, but may be alternatively arranged by, for instance, a computer having a retrieving function based upon a gene expression profile, and another computer having a gene expression profile DB. In this case, gene expression DB may be arranged by several computers which are connected via a network. Also, platforms of gene expression profiles which have been stored in the respective computers may be alternatively different from each other. Further, the gene number converting unit 26 for converting a gene number used in a different platform into a gene number of UniGene may be alternatively provided in the above-described computer having the retrieving function, or may be alternatively provided in respective computers connected to a network. As a result, gene expression files which have been stored in computers installed in research institutions and the like in the world may be utilized and the retrievable range may be enlarged.

Also, in the above-explained embodiment, in order to thoroughly select the genes as the reference genes from the gene having the large order of the expression level up to the gene having the small order of the expression level, the genes have been subdivided into the plural groups according to the orders of the expression level. Alternatively, the genes may be alternatively subdivided into the plural groups according to magnitudes of the expression level.

Also, in the above-described embodiment, the gene expression profile of the known cell may be alternatively employed as the inquiry profile. As a result, since a cell which is highly analogous to the known cell, a relationship between these cells may be clarified, so that the cell sorts may be classified. If the cells can be correctly classified, then the correctly-classified cells may be applied to embryology and medical fields.

As previously explained, the present invention can have a superior effect that the cell which is analogous to the inquiry profile can be retrieved from the gene expression profile database which has stored the gene expression profiles of the cells in the plural different platforms. The present invention is useful as a gene expression profile retrieving apparatus capable of retrieving a cell from a database which stores gene expression profiles of known cells by using a gene expression profile as a retrieving key.

Persons of ordinary skill in the art will realize that many modifications and variations of the above embodiments may be made without departing from the novel and advantageous features of the present invention. Accordingly, all such modifications and variations are intended to be included within the scope of the appended claims. The specification and examples are only exemplary. The following claims define the true scope and spirit of the invention.

What is claimed is:

1. A gene expression profile retrieving apparatus comprising:
a gene expression profile database which stores the gene expression profile of known cells, wherein the profile data have been acquired with a plurality of different platforms;
input section operable to accept an input of an inquiry profile indicative of a gene expression profile of a cell to be retrieved;
reference gene selecting section operable to select a plurality of reference genes from plural genes which are commonly contained in both the platform of said inquiry profile and a platform of the gene expression profile stored in said gene expression profile database;
order applying section operable to apply orders to the reference genes of said inquiry profile according to the expression level of each gene, and to apply orders to the reference genes of each cells stored in said gene expression profile database according to the expression level of each gene;
analogous cell determining section operable to determine a cell from the plural cells stored in said gene expression profile database, wherein a combination of the orders applied to the respective reference genes of the cell to be determined is analogous to a combination of the orders applied to the reference genes of said inquiry profile in the highest degree; and
output section operable to output the cell determined by said analogous cell determining section as a retrieved result.

2. A gene expression profile retrieving apparatus as claimed in claim 1 wherein:
said reference gene selecting section subdivides the genes which constitute said inquiry profile into a plurality of groups according to the expression level of each gene, and selects at least one gene from each groups as said reference gene.

3. A gene expression profile retrieving apparatus as claimed in claim 1 wherein:
said reference gene selecting section selects a predetermined number of genes as said reference genes so that cells can be distinguished from each other based upon an analogous degree of the combinations of said orders.

4. A gene expression profile retrieving apparatus as claimed in claim 1 wherein:
said reference gene selecting section selects 50 or more pieces of genes as said reference genes.

5. A gene expression profile retrieving apparatus as claimed in claim 1 wherein:
said analogous cell determining section determines a plurality of cells in the order of higher analogous degrees between the combination of the orders applied to the respective reference genes of the cells which have been stored in said gene expression profile database, and the orders applied to the respective reference genes of said inquiry profile.

6. A gene expression profile retrieving method for retrieving a cell, while a gene expression profile is employed as a key, from a gene expression profile database which stores the gene expression profile of known cells, wherein the profile data have been acquired with a plurality of different platforms, comprising:
an input step for accepting an input of an inquiry profile indicative of a gene expression profile of a cell to be retrieved;
a reference gene selecting step for selecting a plurality of reference genes from plural genes which are commonly contained in both a platform of said inquiry profile and a platform of a gene expression profile stored in the gene expression profile database;
an order applying step for applying an orders to said reference genes of said inquiry profile according to the expression level of each gene, and for applying orders to said reference genes of each cells stored in said gene expression profile database according to the expression level of each gene;
an analogous cell determining step for determining a cell from the plural cells stored in said gene expression profile database, wherein the orders applied to the respective reference genes of the cell to be determined is analogous to a combination of the orders applied to the reference genes of said inquiry profile in the highest degree; and
an output step for outputting the cell acquired by said analogous cell determining step as a retrieved result.

7. A program for retrieving a cell, while a gene expression profile is employed as a key, from a gene expression profile database which stores the gene expression profile of known cells, wherein the profile data have been acquired with a plurality of different platforms,
said program causes a computer to execute:
an input step for accepting an input of an inquiry profile indicative of the gene expression profile of a cell to be retrieved;
a reference gene selecting step for selecting a plurality of reference genes from plural genes which are commonly contained in both a platform of said inquiry profile and a platform of the gene expression profile stored in said gene expression profile database;
an order applying step for applying an order to the reference gene of said inquiry profile according to the expression level of each gene, and for applying an order to said reference gene of each of the cells stored in the gene expression profile database according to the expression level of the gene;
an analogous cell determining step for determining a cell from the plural cells stored in said gene expression profile database, wherein combination of the orders applied to the respective reference genes of the cell to be determined is analogous to a combination of the orders applied to the reference genes of said inquiry profile in the highest degree; and
an output step for outputting the cell determined by said analogous cell determining step as a retrieved result.
